(19) United States
(12) Patent Application Publication
(10) Pub. No.: US 2015/0257777 A1
(43) Pub. Date: Sep. 17, 2015

(54) Malleable Scalpel System

(71) Applicant: Surgistar, Inc., Vista, CA (US)

(72) Inventors: Jonathan Woodward, Vista, CA (US); Payal Goel, Vista, CA (US)

(21) Appl. No.: 14/642,368

(22) Filed: Mar. 9, 2015

Related U.S. Application Data
(60) Provisional application No. 61/951,359, filed on Mar. 11, 2014.

(51) Int. Cl. A61B 17/3211

(52) U.S. Cl. CPC A61B 17/3211; A61B 2017/00946

(57) ABSTRACT

A scalpel having a sharpened edge and a coupling region that is configured to be removably attached to a handle. At least a portion of the scalpel is made of a material that is malleable such that the shape of the scalpel may be modified to provide a shape that improves accessibility to a surgical site.

### REFERENCE TO PRIORITY DOCUMENT

The present application claims priority to co-pending U.S. Provisional Patent Application Ser. No. 61/951,359 entitled “Malleable Scalpel System”, filed Mar. 11, 2014. Priority to the filing date is claimed and the provisional application is incorporated herein by reference in its entirety.

### BACKGROUND

Scalpels were originally manufactured as solid metal handles with a sharpened end. The scalpel and handle were initially manufactured as one integral unit but manufacturers eventually started manufacturing the scalpel and handle as an attachable/detachable system where the handle could be reused and the scalpel disposed of after use. This type of system is still widely used today.

A limitation of this type of system is that the scalpels are brittle and not malleable. Because of this, the common straight scalpels are difficult to get into certain parts of the anatomy, such as in regions of a patient's mouth for example.

### SUMMARY

This disclosure relates to a feature of malleability on a scalpel for utility in any surgical procedure that requires access to hard-to-reach areas presently difficult with an existing scalpels. The malleable nature of the disclosed scalpel allows the physician to customize the scalpel by bending to suit the individual patient's anatomy, such as for use in oral surgery.

In one aspect, there is disclosed a surgical system, comprising: a scalpel having a sharpened edge, the scalpel further having a coupling region that is configured to be removably attached to a handle, wherein at least a portion of the scalpel is made of a material that is malleable such that the shape of the scalpel may be modified to provide a shape that improves accessibility to a surgical site.

In another aspect, there is disclosed method of using a scalpel, comprising: attaching a scalpel a handle; inserting a scalpel near a region of a patient's body that requires surgery; determining that at least a portion of the scalpel should be re-shaped in order to access the region; and re-shaping at least a portion of the scalpel so that the scalpel can access the region.

Other features and advantages should be apparent from the following description of various embodiments, which illustrate, by way of example, the principles of the invention.

### BRIEF DESCRIPTION OF THE DRAWINGS

FIG. 1 shows an example scalpel system.

FIGS. 2A-2C shows an exemplary scalpel of the scalpel system.

FIGS. 3A-5B shows additional embodiments of scalpels.

### DETAILED DESCRIPTION

Before the present subject matter is further described, it is to be understood that this subject matter described herein is not limited to particular embodiments described, as such may of course vary. It is also to be understood that the terminology used herein is for the purpose of describing a particular embodiment or embodiments only, and is not intended to be limiting. Unless defined otherwise, all technical terms used herein have the same meaning as commonly understood by one skilled in the art to which this subject matter belongs.

Disclosed is a scalpel system that is configured for surgical use such as to form an incision or to dissect through soft tissue. The scalpel system includes a blade that is sized and shaped to be inserted into a surgical site, such as into the mouth of patient. At least a portion of the scalpel is made of a material that is malleable such that the shape of the scalpel and/or the orientation of the scalpel relative to a handle may be modified to provide a shape that improves accessibility to the entire surgical site. This permits a user to selectively modify the shape or orientation of the scalpel to accommodate or fit locations in the surgical site that may be hard to reach. For example, a portion of the scalpel may be modified to have a desired orientation relative to another portion of the scalpel or relative to a scalpel handle. In an embodiment, the scalpel system is configured for use in oral surgery for a human although the scalpel system can be adapted for use in other parts of human or other animal anatomy. In a non-limiting example embodiment, the scalpel is malleable in that a user can bend the malleable material with his or her hands without the need for a separate tool. In a non-limiting example embodiment, the scalpel is malleable in that a user can bend the malleable material using a hand-held tool such a pair of pliers.

In an embodiment, at least 50 percent of the scalpel is made of a malleable material. In another embodiment, no more than 50 percent of the scalpel is made of a malleable material.

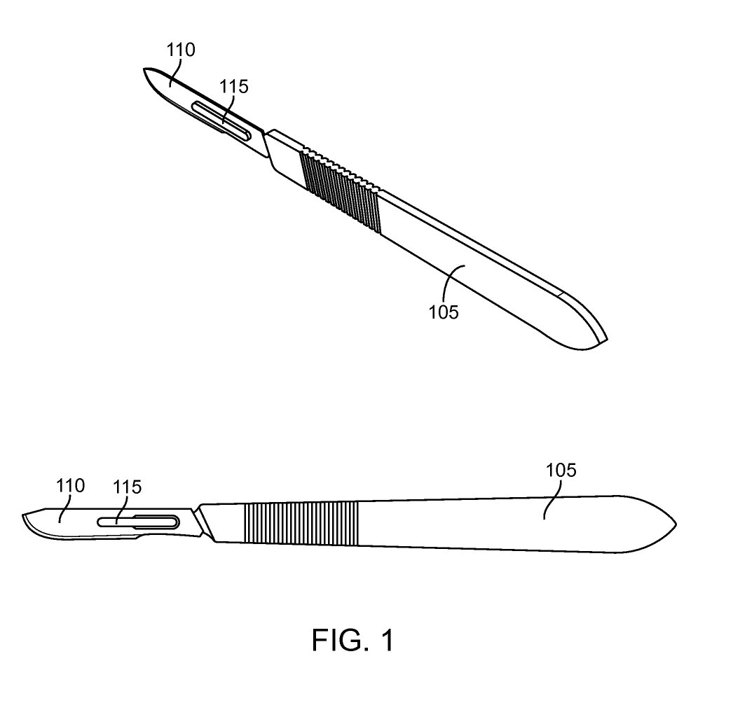

FIG. 1 shows a perspective view of a scalpel system having a handle 105 and a scalpel 110 that is removably attachable to the handle 105. The scalpel 110, which is described in further detail below, has at least one sharpened edge configured to cut through tissue. The handle 105 has an attachment feature 115 where the scalpel 110 may be attached and removed from the handle. It should be appreciated that the particular shape of the handle 105 and the scalpel 110, as well as the overall shape of the scalpel system, may vary. Thus, the scalpel system shown in FIG. 1 is just an example and is not limiting to this disclosure. The attachment feature 115 may vary in structure and can be any type of attachment mechanism that removably attached a scalpel to a handle.

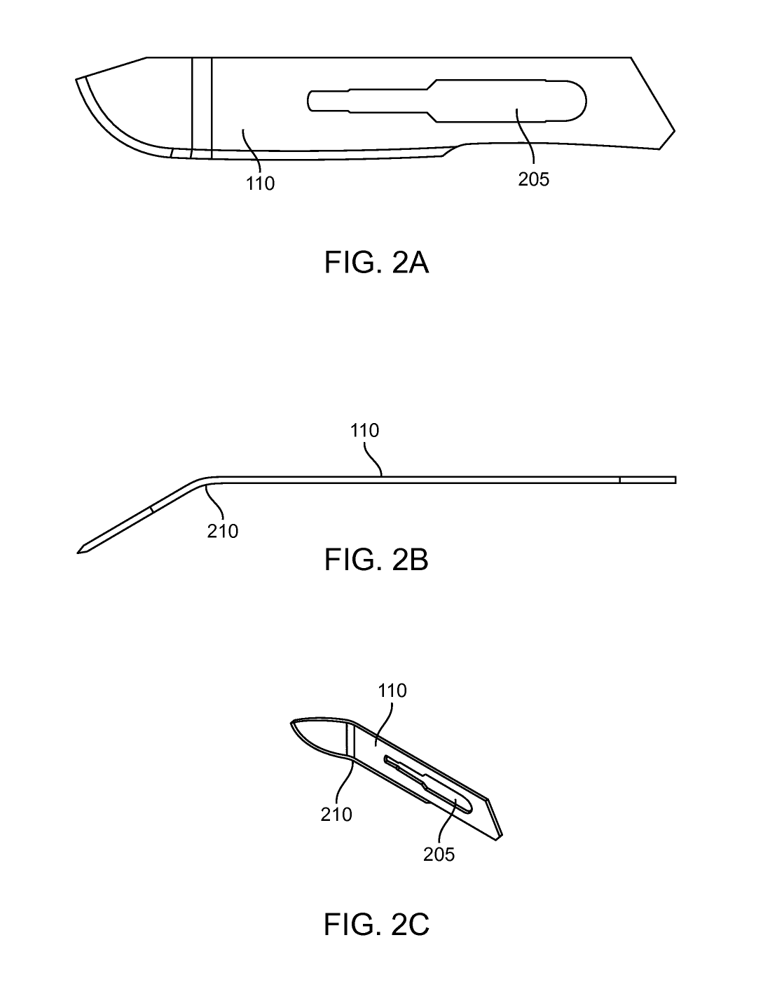

FIGS. 2A-2C shows an exemplary scalpel 110. The scalpel 110 has a coupling region 205 that is configured to be attached to the handle 105. As mentioned, the mechanism of attachment to the handle may vary. In the illustrated embodiment, the coupling region 205 has an opening that is configured to mate with or otherwise couple to a complementary shaped coupling region of the handle 105. In the illustrated embodiment, the scalpel has an elongated shape with a bent region 210 that causes a distal top of the scalpel to bend outward relative to the coupling region 205. That is, the bent region lies along a plane that is non parallel to a plane of the coupling region wherein a bend connects the bent region to the coupling region. This particular default shape, which may vary and which may be varied based on what portion of the scalpel is malleable, is configured to provide the scalpel 110 with a contour that is suited for accessing particular locations of tissue.

As mentioned, the scalpel 110 is at least partially made of a material that is malleable. The whole scalpel may be made of such a material or just a portion of the scalpel may be made of such a material. The use of such a malleable material permits a user to re-shape the contour of the scalpel 110 such as to re-contour the scalpel for improving or revising access of the scalpel to different locations of a surgical site. For example, the user may bend the straight shape to a re-shaped shape such as to create and angle at which the distal region of the scalpel bends away from a handle to which the scalpel is attached. The type of material may vary and may include, for example, 400 series stainless steel.

As mentioned, the portion of the scalpel that is made of a malleable material can be re-shaped relative to a non-malleable portion, if present, of the scalpel. The malleable portion can be re-shaped to be parallel, non-parallel, or a combination thereof relative to another portion of the scalpel.

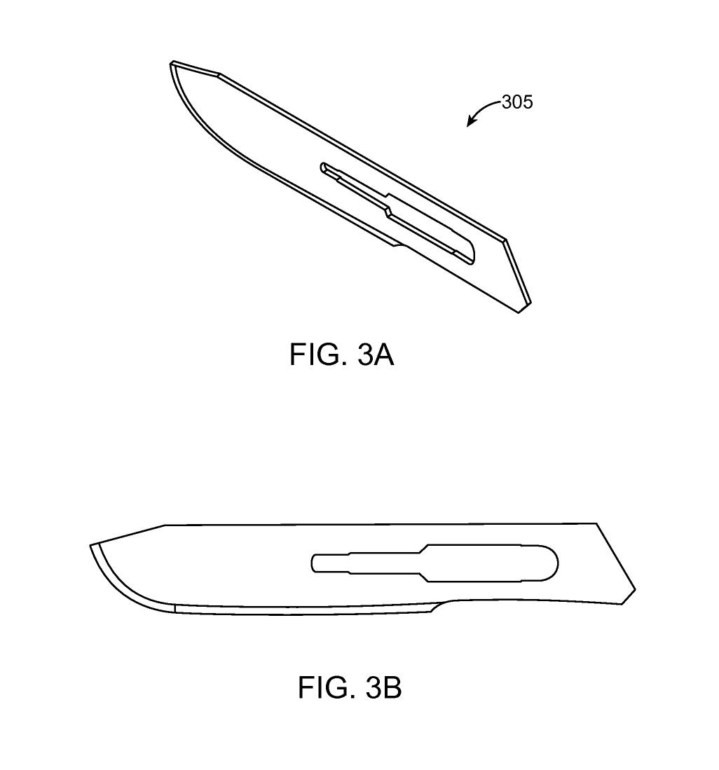

FIG. 3A-5B show additional embodiments of scalpels configured for use with the system. FIG. 3A shows a perspective view of a scalpel 305. FIG. 3B shows a side view of the scalpel 305 including exemplary dimensions in millimeters.

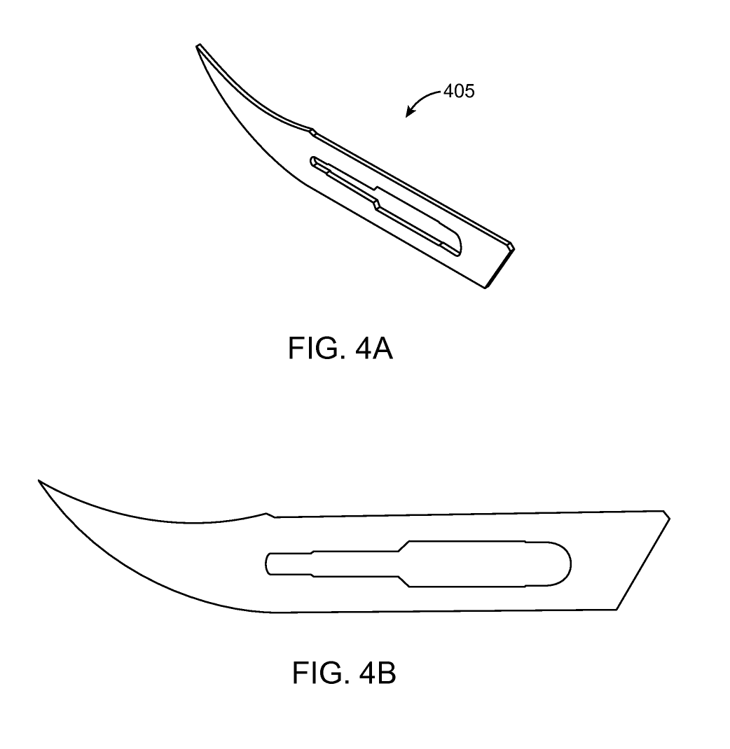

FIG. 4A shows a perspective view of a scalpel 405. FIG. 4B shows a side view of the scalpel 405 including exemplary dimensions in millimeters.

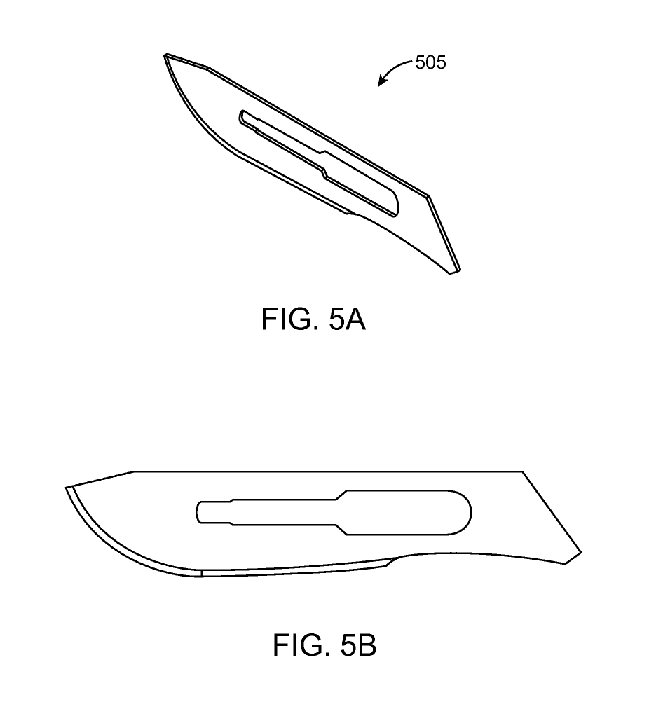

FIG. 5A shows a perspective view of a scalpel 505. FIG. 4B shows a side view of the scalpel 505 including exemplary dimensions in millimeters. It should be appreciated that all the dimensions and shapes for the scalpels shown herein are examples and that deviations are within this disclosure. In an embodiment, the scalpel system or the scalpel 110 is sized and shaped to fit within a human mouth for performing oral surgery.

In a corresponding method, a user attaches the scalpel 110 to the handle. The user then inserts the scalpel into or near a region of a patient's body for surgery, such as into a patient's mouth for cutting through tissue in the patient's mouth. The user can determine that at least a portion of the scalpel should be re-shaped in order to access a certain region of the patient's mouth. The user can then re-shape at least a portion of the scalpel by bending or otherwise re-shaping a malleable portion of the scalpel. The re-shaping can be done using the person's hands or a hand held tool in non-limiting embodiments. The user may also remove the scalpel from the handle and attach a second scalpel to the handle wherein the second scalpel is of a different size or shape relative to the first scalpel. The second scalpel may also have a different percentage of malleable material relative to non-malleable material with respect to the first scalpel.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A surgical system, comprising:
a scalpel having a sharpened edge, the scalpel further having a coupling region that is configured to be removably attached to a handle, wherein at least a portion of the scalpel is made of a material that is malleable such that the shape of the scalpel may be modified to provide a shape that improves accessibility to a surgical site.

2. A surgical scalpel as in claim 1, further comprising a handle that removably attaches to the scalpel.

3. A surgical scalpel as in claim 1, wherein the scalpel is sized and shaped for surgery.

4. A surgical scalpel as in claim 3, wherein the scalpel is sized and shaped for oral surgery in a human.

5. A surgical scalpel as in claim 1, wherein at least 50 percent of the scalpel is made of a malleable material.

6. A surgical scalpel as in claim 1, wherein at least a portion of the scalpel is bent in a default shape.

7. A method of using a scalpel, comprising:
attaching a scalpel a handle;
inserting a scalpel near a region of a patient's body that requires surgery;
determining that at least a portion of the scalpel should be re-shaped in order to access the region; and
re-shaping at least a portion of the scalpel so that the scalpel can access the region.

8. A method as in claim 7, wherein the region of the patient's body is the patient's mouth.

9. A method as in claim 8, further comprising inserting the scalpel into the patient's mouth after the scalpel has been re-shaped.

10. A method as in claim 7, wherein re-shaping at least a portion of the scalpel comprises bending a malleable region of the scalpel with the user's hands.

11. A method as in claim 7, wherein re-shaping at least a portion of the scalpel comprises bending a malleable region of the scalpel with a hand-held tool.
